(19) United States
(12) Patent Application Publication
(10) Pub. No.: US 2011/0065093 A1
(43) Pub. Date: Mar. 17, 2011

(54) NOVEL TOOL FOR THE ANALYSIS OF NEURAL CIRCUITS

(76) Inventors: Kamill Balint, Basel (CH); Zsolt Boldogköi, Szeged (HU); Botond Roska, Oberwil (CH)

(21) Appl. No.: 12/921,865

(22) PCT Filed: Mar. 9, 2009
(86) PCT No.: PCT/EP09/52706

§ 371 Date: Dec. 1, 2010

(30) Foreign Application Priority Data
Mar. 11, 2008 (EP) 08152609.7

(51) Int. Cl. C12Q 1/70; C12N 7/01

(52) U.S. Cl. 435/5; 435/235.1

(57) ABSTRACT

The application relates to an isolated transsynaptic virus expressing an exogenous fluorescent activity sensor. Preferably, the transsynaptic virus is a rhabdovirus, e.g. rabies virus, or a herpesvirus, preferably an alphaherpesvirus, e.g. pseudorabies virus. The fluorescent exogenous activity sensor used in the transsynaptic virus can be a fluorescent protein Ca²⁺ sensor, e.g. yellow cameleon, camgaroo, G-CaMP/Pericam, or TN-L15, preferably TN-L15, or a fluorescent protein voltage sensor, e.g. FlaSh, SPARC, or a VSP, preferably VSP1. The transsynaptic virus of the invention can be used in a method for analyzing the neural activity in local circuits. Moreover, the present application also relates to a kit for analyzing the neural activity in local circuits, said kit comprising a transsynaptic virus of the invention.

### FIELD OF THE INVENTION

The present invention provides novel molecular tools for the analysis of neural circuits.

### BACKGROUND OF THE INVENTION

The mammalian brain incorporates a large number of cell types¹ that are organized into local circuits which perform behaviorally relevant computations. A major challenge in understanding the structure and function of local or more global brain circuits is that in the same nucleus or layered structure like the cortex or the retina the different functional circuits are mixed together and perform parallel computations. There is no brain region where the precise wiring diagram between each neuronal subtype is known which greatly hinders understanding the functional architecture of parallel local circuits.

Transsynaptic rabies² (RV) and pseudorabies³⁻⁶ (PRV) viruses expressing green or red fluorescent proteins were shown to be effective tools to reveal the connected elements within a complex mesh of local neuronal circuits^{2, 7} but these tools can only be used to “highlight” the neurons in a defined circuit in vivo or in vitro.

There is therefore a need in the art for tools which allow understanding the dynamics of neural activity in local circuits by to record activity in the transsynaptically virus-marked neurons.

### SUMMARY OF THE INVENTION

As explained herein-above, there was a desire in the art for means allowing to record activity in the transsynaptically virus-marked neurons. Optical methods using genetically encoded fluorescent activity indicators were known in the art, as reviewed by Knöpfel et al.⁸. However, as explained in the review of Knöpfel et al.⁸, the genetically encoded fluorescent activity indicators known are much larger than conventional (non protein) indicators. In addition, as explained in the review of Knöpfel et al.⁸ as well, the use of virus-based transfection was thought to be associated with considerable uncertainty about the identity of the labeled cells and/or whether the labeled cells are representative for the given target population.

To address this need, and despite the “teaching away” presented herein-above, the present inventors engineered retrograde viruses expressing genetically encoded Calcium indicators (Activity sensor PRV) and were able to show that even 3-4 synapses away from the initial, in vivo injection site, physiological stimulation mediated Ca signals from many neurons can be recorded.

The present invention therefore encompasses an isolated transsynaptic virus expressing an exogenous fluorescent activity sensor. Preferably, the transsynaptic virus is a rhabdovirus, e.g. rabies virus, or a herpesvirus, preferably an alphaherpesvirus, e.g. pseudorabies virus. The fluorescent exogenous activity sensor used in the present invention can be a fluorescent protein Ca²⁺ sensor, e.g. yellow cameleon, camgaroo, G-CaMP/Pericam, or TN-L15, preferably TN-L15, or a fluorescent protein voltage sensor, e.g. FlaSh, SPARC, or a VSP, preferably VSP1.

The present invention also encompasses a method for analyzing the neural activity in local circuits wherein one or more neuron is infected with a transsynaptic virus of the invention and the activity in the transsynaptically virus-marked neurons is analyzed by measuring the fluorescent properties of the fluorescent exogenous activity sensor.

Moreover, the scope of the present invention also includes a kit for analyzing the neural activity in local circuits in a method according to claim 4, said kit comprising a transsynaptic virus of the invention.

### DETAILED DESCRIPTION OF THE INVENTION

As explained herein-above, there was a desire in the art for means allowing to record activity in the transsynaptically virus-marked neurons. Optical methods using genetically encoded fluorescent activity indicators were known in the art, as reviewed by Knöpfel et al.⁸. However, as explained in the review of Knöpfel et al.⁸, the genetically encoded fluorescent activity indicators known are much larger than conventional (non protein) indicators. In addition, as explained in the review of Knöpfel et al.⁸ as well, the use of virus-based transfection was thought to be associated with considerable uncertainty about the identity of the labeled cells and/or whether the labeled cells are representative for the given target population.

To address this need, and despite the “teaching away” presented herein-above, the present inventors engineered retrograde viruses expressing genetically encoded Calcium indicators (Activity sensor PRV) and were able to show that even 3-4 synapses away from the initial, in vivo injection site, physiological stimulation mediated Ca signals from many neurons can be recorded.

The present invention therefore encompasses an isolated transsynaptic virus expressing an exogenous fluorescent activity sensor. Preferably, the transsynaptic virus is a rhabdovirus, e.g. rabies virus, or a herpesvirus, preferably an alphaherpesvirus, e.g. pseudorabies virus. The fluorescent exogenous activity sensor used in the present invention can be a fluorescent protein Ca²⁺ sensor, e.g. yellow cameleon, camgaroo, G-CaMP/Pericam, or TN-L15, preferably TN-L15, or a fluorescent protein voltage sensor, e.g. FlaSh, SPARC, or a VSP, preferably VSP1.

The present invention also encompasses a method for analyzing the neural activity in local circuits wherein one or more neuron is infected with a transsynaptic virus of the invention and the activity in the transsynaptically virus-marked neurons is analyzed by measuring the fluorescent properties of the fluorescent exogenous activity sensor.

Moreover, the scope of the present invention also includes a kit for analyzing the neural activity in local circuits in a method according to claim 4, said kit comprising a transsynaptic virus of the invention.

These and other aspects of the present invention should be apparent to those skilled in the art, from the teachings herein.

For convenience, the meaning of certain terms and phrases employed in the specification, examples, and appended claims are provided below.

The singular forms “a,” “an,” and “the” include plural reference unless the context clearly dictates otherwise.

A “virus” is a sub-microscopic infectious agent that is unable to grow or reproduce outside a host cell. Each viral particle, or virion, consists of genetic material, DNA or RNA, within a protective protein coat called a capsid. The capsid shape varies from simple helical and icosahedral (polyhedral or near-spherical) forms, to more complex structures with tails or an envelope. Viruses infect cellular life forms and are grouped into animal, plant and bacterial types, according to the type of host infected.

The term “transsynaptic virus” as used herein refers to viruses able to migrate from one neurone to another connecting neurone through a synapse. Examples of such transsynaptic virus are rhabodiviruses, e.g. rabies virus, and alphaherpesviruses, e.g. pseudorabies or herpes simplex virus. The term “transsynaptic virus” as used herein also encompasses viral sub-units having by themselves the capacity to migrate from one neurone to another connecting neurone through a synapse and biological vectors, such as modified viruses, incorporating such a sub-unit and demonstrating a capability of migrating from one neurone to another connecting neurone through a synapse.

Transsynaptic migration can be either anterograde or retrograde. During a retrograde migration, a virus will travel from a postsynaptic neuron to a presynaptic one. Accordingly, during anterograde migration, a virus will travel from a presynaptic neuron to a postsynaptic one.

A “fluorescent activity sensor” is a fluorescent protein which will alter its fluorescent properties in response to a signal. For instance Ca²⁺ sensors, e.g. yellow cameleon, camgaroo, G-CaMP/Pericam, or TN-L15 will alter their fluorescent properties in the presence of calcium, whereas fluorescent protein voltage sensors, e.g. FlaSh, SPARC, or a VSP, will alter their fluorescent properties in response to changes in the membrane potential. Preferred sensors are VSP1 and/or TN-L15.

“Polynucleotide” and “nucleic acid”, used interchangeably herein, refer to polymeric forms of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleotide phosphoramidate or a mixed phosphoramidate-phosphodiester oligomer. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids, PNA and LNA. Polynucleotides may further comprise genomic DNA, cDNA, or DNA-RNA hybrids.

“Sequence Identity” refers to a degree of similarity or complementarity. There may be partial identity or complete identity. A partially complementary sequence is one that at least partially inhibits an identical sequence from hybridizing to a target polynucleotide; it is referred to using the functional term “substantially identical.” The inhibition of hybridization of the completely complementary sequence to the target sequence may be examined using a hybridization assay (Southern or Northern blot, solution hybridization and the like) under conditions of low stringency. A substantially identical sequence or probe will compete for and inhibit the binding (i.e., the hybridization) of a completely identical sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarities (e.g., less than about 30% identity); in the absence of non-specific binding, the probe will not hybridize to the second non-complementary target sequence.

Another way of viewing sequence identity in the context to two nucleic acid or polypeptide sequences includes reference to residues in the two sequences that are the same when aligned for maximum correspondence over a specified region. As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

“Gene” refers to a polynucleotide sequence that comprises control and coding sequences necessary for the production of a polypeptide or precursor. The polypeptide can be encoded by a full length coding sequence or by any portion of the coding sequence. A gene may constitute an uninterrupted coding sequence or it may include one or more introns, bound by the appropriate splice junctions. Moreover, a gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. In this regard, such modified genes may be referred to as “variants” of the “native” gene.

“Expression” generally refers to the process by which a polynucleotide sequence undergoes successful transcription and translation such that detectable levels of the amino acid sequence or protein are expressed. In certain contexts herein, expression refers to the production of mRNA. In other contexts, expression refers to the production of protein.

“Cell type” refers to a cell from a given source (e.g., tissue or organ) or a cell in a given state of differentiation, or a cell associated with a given pathology or genetic makeup.

“Polypeptide” and “protein”, used interchangeably herein, refer to a polymeric form of amino acids of any length, which may include translated, untranslated, chemically modified, biochemically modified, and derivatized amino acids. A polypeptide or protein may be naturally occurring, recombinant, or synthetic, or any combination of these. Moreover, a polypeptide or protein may comprise a fragment of a naturally occurring protein or peptide. A polypeptide or protein may be a single molecule or may be a multi-molecular complex. In addition, such polypeptides or proteins may have modified peptide backbones. The terms include fusion proteins, including fusion proteins with a heterologous amino acid sequence, fusions with heterologous and homologous leader sequences, with or without N-terminal methionine residues, immunologically tagged proteins, and the like.

A “fragment of a protein” refers to a protein that is a portion of another protein. For example, fragments of proteins may comprise polypeptides obtained by digesting full-length protein isolated from cultured cells. In one embodiment, a protein fragment comprises at least about 6 amino acids. In another embodiment, the fragment comprises at least about 10 amino acids. In yet another embodiment, the protein fragment comprises at least about 16 amino acids.

An “expression product” or “gene product” is a biomolecule, such as a protein or mRNA, that is produced when a gene in an organism is transcribed or translated or post-translationally modified.

“Host cell” refers to a microorganism, a prokaryotic cell, a eukaryotic cell or cell line cultured as a unicellular entity that may be, or has been, used as a recipient for a recombinant vector or other transfer of polynucleotides, and includes the progeny of the original cell that has been transfected. The progeny of a single cell may not necessarily be completely identical in morphology or in genomic or total DNA complement as the original parent due to natural, accidental, or deliberate mutation.

The term “functional equivalent” is intended to include the “fragments”, “mutants”, “derivatives”, “alleles”, “hybrids”, “variants”, “analogs”, or “chemical derivatives” of the native gene or virus.

“isolated” refers to a polynucleotide, a polypeptide, an immunoglobulin, a virus or a host cell that is in an environment different from that in which the polynucleotide, the polypeptide, the immunoglobulin, the virus or the host cell naturally occurs.

“Substantially purified” refers to a compound that is removed from its natural environment and is at least about 60% free, at least about 65% free, at least about 70% free, at least about 75% free, at least about 80% free, at least about 83% free, at least about 85% free, at least about 88% free, at least about 90% free, at least about 91% free, at least about 92% free, at least about 93% free, at least about 94% free, at least about 95% free, at least about 96% free, at least about 97% free, at least about 98% free, at least about 99% free, at least about 99.9% free, or at least about 99.99% or more free from other components with which it is naturally associated.

“Diagnosis” and “diagnosing” generally includes a determination of a subject's susceptibility to a disease or disorder, a determination as to whether a subject is presently affected by a disease or disorder, a prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

“Biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived therefrom and the progeny thereof. The term specifically encompasses a clinical sample, and further includes cells in cell culture, cell supernatants, cell lysates, serum, plasma, urine, amniotic fluid, biological fluids, and tissue samples. The term also encompasses samples that have been manipulated in any way after procurement, such as treatment with reagents, solubilization, or enrichment for certain components.

“Individual”, “subject”, “host” and “patient”, used interchangeably herein, refer to any mammalian subject for whom diagnosis, treatment, or therapy is desired. In one preferred embodiment, the individual, subject, host, or patient is a human. Other subjects may include, but are not limited to, cattle, horses, dogs, cats, guinea pigs, rabbits, rats, primates, and mice.

“Hybridization” refers to any process by which a polynucleotide sequence binds to a complementary sequence through base pairing, Hybridization conditions can be defined by, for example, the concentrations of salt or formamide in the prehybridization and hybridization solutions, or by the hybridization temperature, and are well known in the art. Hybridization can occur under conditions of various stringency.

“Stringent conditions” refers to conditions under which a probe may hybridize to its target polynucleotide sequence, but to no other sequences. Stringent conditions are sequence-dependent (e.g., longer sequences hybridize specifically at higher temperatures). Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e.g., 10 to 50 nucleotides).

Stringent conditions may also be achieved with the addition of destabilizing agents, such as formamide.

“Biomolecule” includes polynucleotides and polypeptides.

“Biological activity” refers to the biological behavior and effects of a protein or peptide. The biological activity of a protein may be affected at the cellular level and the molecular level. For example, the biological activity of a protein may be affected by changes at the molecular level. For example, an antisense oligonucleotide may prevent translation of a particular mRNA, thereby inhibiting the biological activity of the protein encoded by the mRNA. In addition, an immunoglobulin may bind to a particular protein and inhibit that protein's biological activity.

“Oligonucleotide” refers to a polynucleotide sequence comprising, for example, from about 10 nucleotides (nt) to about 1000 nt. Oligonucleotides for use in the invention are preferably from about 15 nt to about 150 nt, more preferably from about 150 nt to about 1000 nt in length. The oligonucleotide may be a naturally occurring oligonucleotide or a synthetic oligonucleotide.

“Modified oligonucleotide” and “Modified polynucleotide” refer to oligonucleotides or polynucleotides with one or more chemical modifications at the molecular level of the natural molecular structures of all or any of the bases, sugar moieties, internucleoside phosphate linkages, as well as to molecules having added substitutions or a combination of modifications at these sites. The internucleoside phosphate linkages may be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone internucleotide linkages, or 3′-3′, 5′-3′, or 5′-51 linkages, and combinations of such similar linkages. The phosphodiester linkage may be replaced with a substitute linkage, such as phosphorothioate, methylamino, methylphosphonate, phosphoramidate, and guanidine, and the ribose subunit of the polynucleotides may also be substituted (e.g., hexose phosphodiester; peptide nucleic acids). The modifications may be internal (single or repeated) or at the end (s) of the oligonucleotide molecule, and may include additions to the molecule of the internucleoside phosphate linkages, such as deoxyribose and phosphate modifications which cleave or crosslink to the opposite chains or to associated enzymes or other proteins. The terms “modified oligonucleotides” and “modified polynucleotides” also include oligonucleotides or polynucleotides comprising modifications to the sugar moieties (e.g., 3′-substituted ribonucleotides or deoxyribonucleotide monomers), any of which are bound together via 5′ to 3′ linkages.

“Biomolecular sequence” or “sequence” refers to all or a portion of a polynucleotide or polypeptide sequence.

The term “detectable” refers to a polynucleotide expression pattern which is detectable via the standard techniques of polymerase chain reaction (PCR), reverse transcriptase- (RT) PCR, differential display, and Northern analyses, which are well known to those of skill in the art. Similarly, polypeptide expression patterns may be “detected” via standard techniques including immunoassays such as Western blots.

A “target gene” refers to a polynucleotide, often derived from a biological sample, to which an oligonucleotide probe is designed to specifically hybridize. It is either the presence or absence of the target polynucleotide that is to be detected, or the amount of the target polynucleotide that is to be quantified. The target polynucleotide has a sequence that is complementary to the polynucleotide sequence of the corresponding probe directed to the target. The target polynucleotide may also refer to the specific subsequence of a larger polynucleotide to which the probe is directed or to the overall sequence (e.g., gene or mRNA) whose expression level it is desired to detect.

A “target protein” refers to a polypeptide, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein or amino acid may also refer to the specific substructure of a larger protein to which the protein-capture agent is directed or to the overall structure (e.g., gene or mRNA) whose expression level it is desired to detect.

“Complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like.

The term “fusion protein” refers to a protein composed of two or more polypeptides that, although typically not joined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological conditions” means conditions that are typical inside a living organism or a cell. Although some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. The concentration of various salts depends on the organ, organism, cell, or cellular compartment used as a reference.

“BLAST” refers to Basic Local Alignment Search Tool, a technique for detecting ungapped sub-sequences that match a given query sequence.

“BLASTP” is a BLAST program that compares an amino acid query sequence against a protein sequence database. “BLASTX” is a BLAST program that compares the six-frame conceptual translation products of a nucleotide query sequence (both strands) against a protein sequence database.

A “cds” is used in a GenBank DNA sequence entry to refer to the coding sequence. A coding sequence is a sub-sequence of a DNA sequence that is surmised to encode a gene.

A “consensus” or “contig sequence”, as understood herein, is a group of assembled overlapping sequences, particularly between sequences in one or more of the databases of the invention.

The sensors as used in the present invention will be produced by a virus harbouring a nucleic acid that encodes the sensor gene sequence. The virus may comprise elements capable of controlling and/or enhancing expression of the nucleic acid. The virus may be a recombinant virus. The recombinant virus may also include other functional elements. For instance, recombinant viruses can be designed such that the viruses will autonomously replicate in the target cell. In this case, elements that induce nucleic acid replication may be required in a recombinant virus. The recombinant virus may also comprise a promoter or regulator or enhancer to control expression of the nucleic acid as required. Tissue specific promoter/enhancer elements may be used to regulate expression of the nucleic acid in specific cell types. The promoter may be constitutive or inducible.

Contaminant components of its natural environment are materials that would interfere with the viruses of the invention, and may include enzymes, hormones, and other proteinaceous or nonproteinaceous solutes. Ordinarily, an isolated agent will be prepared by at least one purification step. In one embodiment, the agent is purified to at least about 60%, at least about 65%, at least about 70%, at least about 75%, at least about 80%, at least about 85%, at least about 88%, at least about 90%, at least about 92%, at least about 95%, at least about 97%, at least about 98%, at least about 99%, at least about 99.9%, or at least about 99.99% by weight of e.g. virus.

In an alternative invention, the fluorescent activity sensor will be introduced into cells in a manner well known in the art under the control of a specific promoter and the promoter will be activated by a ligand brought into the cell by a transsynaptic virus.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Construction of Recombinant Viruses

Recombinant PRV strains were constructed by insertion of exogenous DNA sequences to the viral genome via homologous recombination. Other techniques for genetic manipulation of viral genome, including site specific recombination (Sauer B at al., Proc Natl Acad Sci USA, 1988, 85, 5166-5170), restriction cleavage/ligation (Boldogköi Z at al., Res Virol, 1998, 149, 87-97) have also been described. The first step of homologous recombination-based techniques requires the subcloning of PRV sequences, which includes the target site. Subsequently, the exogenous DNA sequences, for example a fluorescent reporter gene expression cassette is inserted to an internal position of virus-derived DNA resulting in the generation of the targeting plasmid. The two arms bracketing the foreign DNA (termed flanking sequences) in the targeting plasmid serves as homologous sequences for the recombination with the viral genome, which results in the insertion of desired exogenous gene to the PRV genome. A palindrome-containing plasmids positive-selection vector (pRL; Elhai J and Wolk C P, Gene 1988, 68, 119-138) family was used for the generation of targeting plasmids. Foreign DNA insertion to the PRV genome was achieved by transfecting of purified viral DNA along with the linearized targeting construct to porcine kidney (PK-15) cells as described by Boldogkoi et al. (J Gen Wok 2000, 81, 415-420). Transfection was performed either by electroporation or by a cationic lipid-based method. Recombinant viruses were screened on the basis of infected cells, which was produced by the inserted marker gene. Subsequently, recombinant viruses were plaque purified and underwent testing for the correct insertion of fluorescent marker genes.

Plaques formed by recombinant viruses carrying the transgenes were detected visually on the basis of their fluorescence. Recombinant viruses were isolated by 6-15 cycles of plaque purification procedure using a fluorescence microscope (Olympus).

More precisely, targeting plasmids were constructed by the insertion of appropriate marker genes to particular locations of various cloned viral DNA sequences according to the following methods:

### Flanking Sequences

The specific restriction fragments of PRV DNA used as flanking sequences were subcloned to members of a palindrome containing positive-selection vector family (pRL479, pRL525, pRL494; Elhai and Wok, 1988). Subsequently, EcoRI linker was inserted to an internal position of PRV sequences, which served as a cloning site for incorporation of marker gene expression cassettes. Table 1 shows the PRV genomic regions that were utilized as flanking sequences in this study.

ASP (putative antisense promoter) region The BamHI-8′ PRV DNA fragment was isolated and subcloned pRL525 vector resulting in p525-B8′. The DraI site of this PRV sequence was replaced by EcoRI generating pASP-RI. The various marker gene expression cassettes were inserted to the EcoRI site of pASP-RI resulting in the generation of ASP-based targeting constructs. Alternatively, BamHI-8′ fragment was subcloned to pRL479 (p479-B8′), followed by converting the DraI site to EcoRI resulting in pASP-HIII.

gE+gI (glycoprotein E and I gene) region The BamHI-7 PRV DNA fragment was isolated and subcloned pRL525 vector generating p525-B7. The 1855-bp StuI-AgeI DNA fragment of p525-B7 was replaced by an EcoRI linker resulting in pB7ΔgEgI-RI. Removal of StuI-AgeI DNA fragment resulted in the inactivation of both gE and gI gene of the virus.

VHS (virion host-shut-off gene) region The 2526-bp XhoI DNA fragment containing the entire vhs gene was subcloned to the SalI site (compatible ends!) of pRL494 resulting in p494-Xh. The unique NruI site of this DNA segment was converted to EcoRI site (generating pVHS-RI) by linker insertion, which resulted a frameshift mutation in the vhs gene.

EP0 (early protein 0 gene) region The KpnI-F fragment of PRV was inserted to pRL525 (p525-KF) followed by the deletion of 1388-bp BamHI fragment containing the entire ep0 gene. The BamHI site was replaced by an EcoRI site via Klenow filling and linker insertion resulting in pEP0Δ.

Ribonucleotide reductase (rr) The SalI fragment of PRV containing both subunits of rr gene was inserted to pRL494 (p494-S2). Subsequently, an EcoRI linker was inserted to the ScaI site of p494-S2 resulting in the generation of pRR2-RI. The linker insertion inactivated the small subunit (rr2) of rr gene.

TABLE 1

| | PRV DNA | Insertion/ |
|---|---|---|
| Region | fragment | deletion site |
| Putative antisense promoter (ASP) | BamHI-8′ | DraI, ins |
| Virion host shut-off gene (vhs) | XhoI | NruI, ins |
| Early protein 0 gene (ep0) | KpnI-F + | BamHI + StuI, |
| | BamHI-8′* | Δ4283 bp |
| Glycoprotein E & I genes (gE + gI) | BamHI-7 | StuI + AgeI, |
| | | Δ1855 bp |
| Ribonucleotide reductase (rr) | BamHI-2 | ScaI, ins |

ins: insertion;
Δdeletion;
*deletion of gE and gI genes was performed in multiple steps using KpnI-F + BamHI-8′ DNA fragments.

### Reporter Genes

Each expression cassette was modified to contain EcoRI restriction endonuclease sites at both ends for their easy subcloning to flanking viral sequences.

The IacZ gene expression cassettes were constructed by using pCMVβ (Amersham) as a parent plasmid. pCMVβ-RI was generated by converting the unique HindIII site of pCMVβ to EcoRI by linker insertion. Conversely, pCMVβ-HIII was constructed by replacing pCMVβ EcoRI site to HindIII site by linker insertion. The resulting plasmids contain either EcoRI or HindIII sites at both ends of the CMVP-IacZ-polyA cassette.

Fluorescent reporter genes pDsRed-2 (Clontech) was used as parent plasmid for the generation of each fluorescent protein gene. As a first step, the AseI site of pDsRed locating upstream of CMVP was converted to EcoRI resulting in pDsRed-RI. Subsequently, this plasmid was further modified by replacing AflII site located downstream of polyA signal of the expression cassette to EcoRI resulting in pDsRed-2xRI. For the generation of pmemGFP the XhoI-NotI fragment of pULmEB was inserted to the XhoI-NotI site of pDsRed-2xRI. As a result of the above cloning protocol, the pDsRed2 cassette was replaced by memGFP and a large part of the molecular cloning site (MCS) of pDsRed-2 was deleted. All the other reporter gene expression cassettes were constructed similarly with the exception that they were cut with BamHI+NotI followed by inserting this restriction fragment to the BglII−NotI site of pDsRed-2xRI. The marker genes and of there origin is indicated in Table Y.

### Activity Markers

pVAMP-GFP was first modified to remove a unique EcoRI site by Klenow filling and religation generating pVAMP-GFP-ΔRI, followed by replacing the XhoI site to NotI by linker insertion resulting in pVAMP-GFP-NΔRI. This latter modification made allow to cut a NotI fragment containing the entire VAMP-GFP gene and insert it to the NotI site of pCMVβ-RI. As a result of this cloning protocol, the lacZ gene of pCMVβ-RI was replaced by VAMP-GFP, thereby equipping this latter gene with promoter (CMVP), polyA signal plus transcription termination sequences and two EcoRI sites bracketing the expression cassette.

Troponeon TNL15 was used to subclone an NruI−PvuI DNA fragment containing the troponeon expression cassette to pASP-RI site generating pASP-tropo.

### Targeting Vectors

The various targeting vectors were constructed by insertion of marker gene expression cassettes to a particular site of flanking sequences (Table 1).

### PRV Injection

PRV strains were harvested from the PK-15 cell line as described earlier³. For AntC injection 8-14 weeks old C57BL/6J mice were injected with 10³-10⁵ plaque forming units of PRV152 in one microliter Dulbecco's Modified Eagle Medium (DMEM) into the AntC of the right eye under isoflurane anesthesia (2% in oxygen). The cornea was punctured with a 27-28 gauge needle and PRV152 was injected into the AntC using a ten microliter Hamilton syringe fitted with a 33 gauge needle. The injection of PRVs into V1 and LGN was done by stereotaxic surgery, 10³-10⁴ plaque forming units of PRV in one hundred nanoliter DMEM was injected to either V1 or LGN. Animals were kept in the same laboratory for up to 150 hours after injection. All the PRV work was done in a biosafety level 2 laboratory. Animal experiments have been approved by the local Institutional Animal Care and Use Committee at the Friedrich Miescher Institute. Animal experiments have been conducted according to the guidelines of the National Institute of Health, US and the Swiss National Foundation, Switzerland.

### Confocal Microscopy and Analysis

The 405, 488 and 543 nm laser lines of a Zeiss LSM 510 Meta confocal microscope were used to excite DAPI, Alexa 488 and Alexa 633 respectively. 63×, 1.4 numerical aperture oil immersion lens (Zeiss) was used. The z steps were 0.2-0.35 microns. The scan started at the ganglion cell layer and continued until the photoreceptor layer.

### Electrophysiology

2-6 days after PRV152 injection into the AntC of the right eye or V1 or LGN, the eyes was removed and the retina was dissected. The isolated retina was continuously superfused at 1 ml/min with Ames (pH 7.4) solution at 36° C. and equilibrated with 95% O2 and 5% CO2. We patch clamp recorded virus labeled ganglion cells in the current clamp configuration with electrodes (5-8 MW) filled with (in mM): 115 KGluconate, 9.7 KCl, 1 MgCl2, 0.5 CaCl2, 1.5 EGTA, 10 HEPES, 4 ATP-Na2, 0.5 GTP-Na3 at pH 7.2 using a Multiclamp 700B patch clamp amplifier (Axon instruments). Virus labeled ganglion cells were visualized by two-photon laser confocal imaging. The visual stimulation and data acquisition software was written in LabView (National Instruments) by David Balya and Thomas Münch. Data were analyzed in ((Mathematica) or Matlab. Visual stimuli were applied using a digital light processor (DLP) projector through the lamp port of an Olympus BX51 upright microscope. The image was focused on the photoreceptors through the condenser before the experiment.

### Two-Photon Imaging

Two-photon laser scanning confocal microscope was used to visualize GFP labeled neurons in the retina in order to avoid bleaching of the photoreceptors. A 950-1020 nm laser line from a Mai Tai HP two photon laser (Spectra Physics) was attenuated by polarization optics and a Pockels cell (Conoptics, Model 302), and was scanned using mirrors (Cambridge Technologies) mounted on a modified Olympus BX51 upright microscope. The two photon microscope was developed by Jens Duebel. The laser energy at the position of the retina was 5-20 mW. The CFP and YFP TN-L15 signal was detected with two PMTs as described previously^{9, 17}. The acquisition software, was written by David Balya. For time-lapse imaging the retina was superfused (as described above) for one day, and at every hour or half hour a z-stack of 80 images was acquired with 1 micron spacings. At the end of the time-lapse imaging the retina was fixed in 4% paraformaldehyde in PBS and stained with antibodies to visualize the circuit at higher resolution.

### Cell Culture Experiments

All cell culture experiments were performed on pig kidney (PK15) fibroblast cells. To define the relative intensities of the different As-PRV strains, we measured the fluorescence intensity of the troponeon in 50 labeled cell/strain 24 h post infection. The samples were excited with a 2-photon laser at 950 nm, the emission intensity was measured at 525/50. Recording of fluorescent time laps curves of the T-PRV were carried out with a wild field fluorescent microscope (Zeiss Long Run, TILLS, axiovert 200 m). We captured the green and red emission in every 20 minutes up to 14 hours post infection or post transfection. Image analysis was made via ImageJ software.

Thus, the inventors developed retrograde, transsynaptic tracers that express strongly and stably genetically encoded exogenous fluorescent activity sensor, e.g. Ca-sensors. One of the Ca indicator described herein above, the a troponin based CFP/YFP-FRET sensor TN-L15⁹, has a ratio metric signal detection that allows expression level independent estimation of Ca levels, so that algorithms well-known to the skilled person can convert the measured dynamics of fluorescent signals to Ca dynamics¹⁰.

To asses if TN-L15 expressed in neurons from As-PRV is able to detect Ca changes a number of synapses away from the initial injection site, the inventors injected one of the generated As-PRV, As-PRV-7, to the right AntC and tested ganglion cells in the contralateral eye. To reach these ganglion cells in the contralateral eye, the virus had to travel through at least 3 synapses. The labeled ganglion cells through this pathway are intrinsically photoreceptive (ipRGCs). ipRGCs are involved both in the pupillary reflex and in the circadian entrainment pathway. Application of ionomycin or glutamate evoked strong changes in FRET signal measured with a two photon microscope in all ganglion cells tested. The inventors then patch-clamped virus labeled ganglion cells and injected depolarizing currents to evoke spiking activity. Strong increase of the fluorescence ratio was observed during neuronal stimulation. Next, the present inventors used physiological stimulation, light, to activate the ganglion cells and found that brief (500 msec) pulse of light evokes strong fluorescent ratio changes. All ganglion cells tested (n=25) showed strong light evoked fluorescent changes. Recording of light evoked activity from many ganglion cells was possible from the same retina demonstrating that As-PRV, e.g. As-PRV-7, allows high throughput activity recordings from well defined cell populations in a neuronal circuit, in this case from ipRGCs of the pupillary reflex and circadian rhythm circuit. These results suggest that neural activity evoked by pharmacological, electrical of physiological light stimulation can be recorded optically, with the help of transsynaptic viruses of the invention, e.g. As-PRV-7, delivered genetically encoded Ca sensor, several synapses away from the injection site.

### REFERENCES

1. Nelson, S. B., Sugino, K. & Hempel, C. M. The problem of neuronal cell types: a physiological genomics approach. Trends Neurosci 29, 339-45 (2006).
2. Wickersham, I. R. et al. Monosynaptic restriction of transsynaptic tracing from single, genetically targeted neurons. Neuron 53, 639-47 (2007).
3. Smith, B. N. et al. Pseudorabies virus expressing enhanced green fluorescent protein: A tool for in vitro electrophysiological analysis of transsynaptically labeled neurons in identified central nervous system circuits. Proc Natl Acad Sci USA 97, 9264-9 (2000).
4. Jons, A. & Mettenleiter, T. C. Green fluorescent protein expressed by recombinant pseudorabies virus as an in vivo marker for viral replication. J Virol Methods 66, 283-92 (1997).
5. Boldogkoi, Z., Erdelyi, F., Sik, A., Freund, T. F. & Fodor, I. Construction of a recombinant herpesvirus expressing the jellyfish green fluorescent protein. Luminescence 14, 69-74 (1999).
6. Banfield, B. W., Kaufman, J. D., Randall, J. A. & Pickard, G. E. Development of pseudorabies virus strains expressing red fluorescent proteins: new tools for multisynaptic labeling applications. J Virol 77, 10106-12 (2003).
7. Viney, T. J. et al. Local retinal circuits of melanopsin-containing ganglion cells identified by transsynaptic viral tracing. Curr Biol 17, 981-8 (2007).
8. Knopfel, T., Diez-Garcia, J. & Akemann, W. Optical probing of neuronal circuit dynamics: genetically encoded versus classical fluorescent sensors. Trends Neurosci 29, 160-6 (2006).
9. Heim, N. & Griesbeck, O. Genetically encoded indicators of cellular calcium dynamics based on troponin C and green fluorescent protein. J Biol Chem 279, 14280-6 (2004).
10. Tay, L. H., Griesbeck, O. & Yue, D. T. Live-Cell Transforms between Ca2+ Transients and FRET Responses for a Troponin-C-Based Ca2+ Sensor. Biophys J (2007).
11. Bertha, A. Experimental reduction of virulence of Aujeszky's disease (in Hungarian). Mag. Ao. Lapja 16, 42-45 (1961).
12. Pickard, G. E. et al. Intravitreal injection of the attenuated pseudorabies virus PRV Bertha results in infection of the hamster suprachiasmatic nucleus only by retrograde transsynaptic transport via autonomic circuits. J Neurosci 22, 2701-10 (2002).
13. Kaplan, A. S. & Vatter, A. E. A comparison of herpes simplex and pseudorabies viruses. Virology 7, 394-407 (1959).
14. Husak, P. J., Kuo, T. & Enquist, L. W. Pseudorabies virus membrane proteins gI and gE facilitate anterograde spread of infection in projection-specific neurons in the rat. J Virol 74, 10975-83 (2000).
15. Campbell, R. E. et al. A monomeric red fluorescent protein. Proc Natl Acad Sci USA 99, 7877-82 (2002).
16. De Paola, V., Arber, S. & Caroni, P. AMPA receptors regulate dynamic equilibrium of presynaptic terminals in mature hippocampal networks. Nat Neurosci 6, 491-500 (2003).
17. Heim, N. et al. Improved calcium imaging in transgenic mice expressing a troponin C-based biosensor. Nat Methods 4, 127-9 (2007).

1. An isolated transsynaptic virus expressing an exogenous fluorescent activity sensor.

2. The transsynaptic virus of claim 1 wherein the transsynaptic virus is a rhabdovirus, e.g. rabies virus, or a herpesvirus, preferably an alphaherpesvirus, e.g. pseudorabies virus.

3. The transsynaptic virus of claim 1 wherein the fluorescent exogenous activity sensor is a fluorescent protein Ca²⁺ sensor, e.g. yellow cameleon, camgaroo, G-CaMP/Pericam, or TN-L15, preferably TN-L15, or a fluorescent protein voltage sensor, e.g. FlaSh, SPARC, or a VSP, preferably VSP1.

4. (canceled)

5. (canceled)

6. A method for analyzing the neural activity in local circuits, the method comprising
infecting one or more neuron with the transsynaptic virus of claim 1, and
measuring the fluorescent properties of the fluorescent exogenous activity sensor expressed by the transsynaptically virus-marked neurons.

7. A kit comprising the transsynaptic virus of claim 1.
